(19) United States
(12) Patent Application Publication
(10) Pub. No.: US 2013/0096397 A1
(43) Pub. Date: Apr. 18, 2013

(54) SENSITIVITY EVALUATION SYSTEM, SENSITIVITY EVALUATION METHOD, AND PROGRAM

(71) Applicants: Hiroaki Kiso, Tokyo (JP); Shinichi Fukuzumi, Tokyo (JP); Keiko Kasamatsu, Tokyo (JP); Hideo Jingu, Ishikawa (JP); Misako Yamagishi, Ishikawa (JP)

(72) Inventors: Hiroaki Kiso, Tokyo (JP); Shinichi Fukuzumi, Tokyo (JP); Keiko Kasamatsu, Tokyo (JP); Hideo Jingu, Ishikawa (JP); Misako Yamagishi, Ishikawa (JP)

(73) Assignee: NEC CORPORATION, Tokyo (JP)

(21) Appl. No.: 13/703,850

(22) PCT Filed: Jun. 16, 2011
(86) PCT No.: PCT/JP2011/064323

§ 371 Date: Dec. 12, 2012

(30) Foreign Application Priority Data
Jun. 17, 2010 (JP) 2010-138556

(51) Int. Cl. A61B 5/00; A61B 5/11; A61B 5/0476; A61B 7/00; A61B 3/11; A61B 5/01

(52) U.S. Cl. CPC A61B 5/4035; A61B 3/112; A61B 5/015; A61B 5/0476; A61B 5/0077; A61B 7/00; A61B 5/1123; A61B 5/7225
USPC 600/301; 600/300; 600/558; 600/549; 600/544; 600/407; 600/586; 600/595

(57) ABSTRACT

Provided is a sensitivity evaluation system for estimating sensitivity of a subject from bio-information with high accuracy, including: an acquisition unit to acquire as biological data, a reaction of a sympathetic nervous system and a reaction of a parasympathetic nervous system; a biological data analysis unit to judge from the obtained biological data, a candidate group of sensitivity factors that the subject has; a positive/negative analysis unit to judge from information obtained from the subject, whether an inner state of the subject is pleasant or unpleasant; and a comprehensive evaluation unit to integrat analysis results of the biological data analysis unit and the positive/negative analysis unit to estimate the sensitivity comprehensively.

### TECHNICAL FIELD

This invention relates to a sensitivity evaluation system, a sensitivity evaluation method, and a program for evaluating sensitivity that a subject has.

### BACKGROUND ART

In a conventional common sensitivity evaluation method, subjective evaluation using a questionnaire mainly consisting of adjectives is used. However, the subjective evaluation method has problems in that the intention of a subject is contained and that potential sensitivity and an intensity thereof are hard to extract.

Prior-art technologies addressing the above-mentioned problems include, for example, the following patent literatures.

As a first prior-art technology, the usability evaluation device according to Patent Literature 1 is described. The usability evaluation device includes the operation inputting part, the biological signal detecting part for detecting a brain wave, and the like. The usability evaluation device acquires an operation performed by the subject in response to an operation instruction to operate the evaluation target and measures a reaction of the subject to the operation result via the brain wave, to thereby use the sensitivity and the intensity thereof felt by the subject through the operation of the evaluation target for the usability evaluation. More specifically, the function evaluating part acquires, for the purpose of utilizing the fact that the brain wave changes in time series depending on the inner state of the subject, the event-related electric potential from the brain wave after the operation of the evaluation target, and evaluates, from the degree of change of the amplitude and the latent time of the electric potential, the easiness to learn, the degree of interest, and the level of proficiency for each function.

As a second prior-art technology, the usability evaluation device according to Patent Literature 2 is described. The usability evaluation device measures a brain wave as a biological signal of the subject as in Patent Literature 1. The usability evaluation device detects, from the detected brain wave, the presence/absence of a disappointment signal (brain wave signal characteristic of when the expectation was not met) in a predetermined time range after the operation of the evaluation target, and in the understanding level judging part, judges the understanding level of the user for the operation of equipment based on the presence/absence of the disappointment signal and the correctness/incorrectness of the user operation obtained from the correct/incorrect operation judging part.

As a third prior-art technology, the bio-information processing apparatus according to Patent Literature 3 is described. The bio-information processing apparatus measures bio-information of the subject in a non-contact manner and in a nonrestricted manner by using image capturing by a camera, and judges a psychological state and its intensity of the subject from the measured value. Further, the bio-information processing apparatus has the operation of the apparatus defined therein in advance in association with the read psychological state of the subject, and operates automatically based on the judged result.

As a fourth prior-art technology, a mental condition judging apparatus according to Patent Literature 4 is described. The mental condition judging apparatus acquires physiological information and voice excitement degree information regarding the excitement of the subject, and judges the mental condition of the subject based on a prestored table of correspondences of mental conditions with respect to relationships of the physiological information and the voice excitement degree information. Further, Patent Literature 4 describes a method of acquiring various kinds of bio-information. Note that, the voice excitement degree information may be regarded as a kind of the bio-information reflecting unconsciousness of the subject.

### CITATION LIST

Patent Document 1: Japanese Unexamined Patent Application Publication (JP-A) No. 2007-052601

Patent Document 2: Japanese Unexamined Patent Application Publication (JP-A) No. 2006-023835

Patent Document 3: Japanese Unexamined Patent Application Publication (JP-A) No. 2006-115865

Patent Document 4: Japanese Unexamined Patent Application Publication (JP-A) No. 2007-296169

### DISCLOSURE OF THE INVENTION

### Problems to be Solved by the Invention

Of sensitivity evaluation methods, the conventional common subjective evaluation method has the problems in that the intention of a subject is contained and that the potential sensitivity and the intensity thereof are hard to extract. The evaluation methods of the above-mentioned prior-art technologies using biological data, which have been proposed as solutions to the problems, need to be improved in accuracy, or require as a precondition the use of an evaluation apparatus having low portability or the measurement while restricting the posture and movement of the subject, which restricts the environment in which the evaluation is performed.

An example of the problems to be improved is that the above-mentioned prior-art technologies acquire the biological data and then judge the sensitivity unambiguously from the acquired data, which leads to a problem in accuracy. This means that, even when the subject has potentially different emotions, the biological sensor may only acquire similar biological data, which remains to be addressed.

This invention provides a portable sensitivity evaluation system for extracting, by using a subject sensitivity evaluation method, the potential sensitivity and the intensity thereof with higher accuracy than in the existing technologies.

### Means to Solve the Problem

According to this invention, there is provided a sensitivity evaluation system, including: an acquisition unit to acquire as biological data, a reaction of a sympathetic nervous system and a reaction of a parasympathetic nervous system; a biological data analysis unit to judge from the obtained biological data, a candidate group of sensitivity factors that a subject has; a positive/negative analysis unit to judge from information obtained from the subject, whether an inner state of the subject is pleasant or unpleasant; and a comprehensive evaluation unit to integrate analysis results of the biological data analysis unit and the positive/negative analysis unit to estimate sensitivity comprehensively.

### Effect of the Invention

According to this invention, it is possible to provide the portable sensitivity evaluation system for extracting, by using the subject sensitivity evaluation method, the potential sensitivity and the intensity thereof with higher accuracy than in the existing technologies.

### BRIEF DESCRIPTION OF THE DRAWING

FIG. 1 is a block diagram illustrating a configuration according to a first embodiment.

FIG. 2 is a flowchart illustrating a flow of processing according to the first embodiment.

FIG. 3 is a block diagram illustrating a configuration according to a second embodiment.

FIG. 4 is a block diagram illustrating another configuration according to the second embodiment.

FIG. 5 is an explanatory diagram illustrating reliability judgment of positive/negative judgment according to the second embodiment.

FIG. 6 is a block diagram illustrating a configuration according to a third embodiment.

FIGS. 7A to 7C are explanatory diagrams illustrating relationships between candidate groups of sensitivity factors and positive or negative judgment results according to an example.

FIGS. 8A to 8C are explanatory diagrams illustrating relationships between a plurality of candidate groups of sensitivity factors and positive and negative judgment results according to the example.

### MODE(S) FOR CARRYING OUT THE INVENTION

Next, modes for carrying out the invention are described in detail with reference to the drawings.

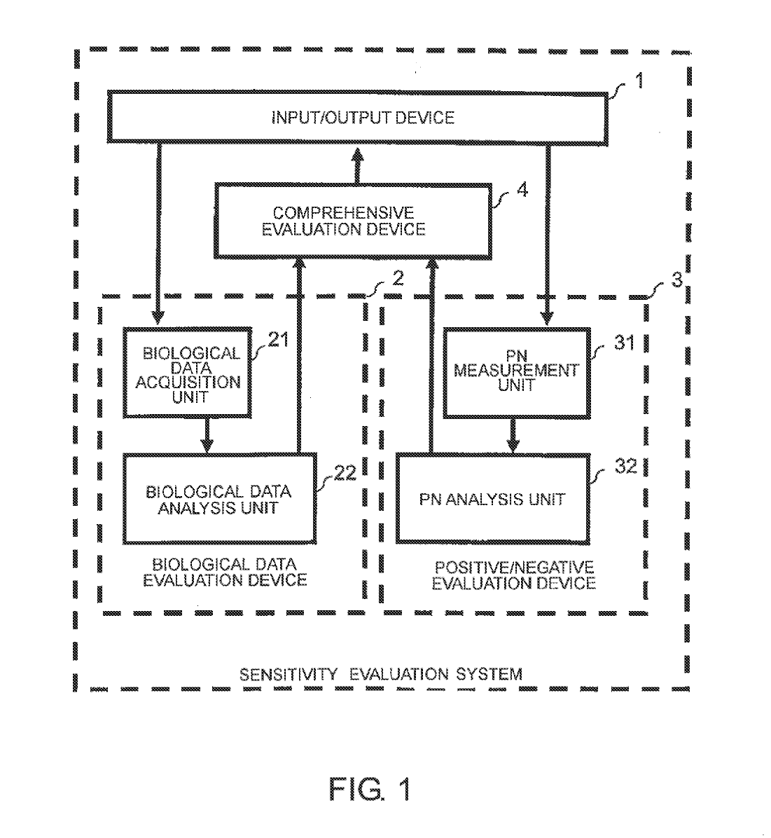

FIG. 1 is a block diagram illustrating a configuration of a sensitivity evaluation system according to an embodiment.

Referring to FIG. 1, the sensitivity evaluation system includes an input/output device 1, a biological data evaluation device 2, a positive/negative evaluation device 3, and a comprehensive evaluation device 4.

The input/output device 1 includes input means such as a mouse and a keyboard, and output means such as a printer and a display for displaying a result output from the comprehensive evaluation device 4. The input/output device 1 also includes sensors, a camera, a microphone, and the like for acquiring autonomic biological data from a subject.

The biological data evaluation device 2 includes a biological data acquisition unit 21 and a biological data analysis unit 22.

The biological data acquisition unit 21 includes an interface circuit for capturing bio-information including information on reactions (such as activities) of a sympathetic nervous system and a parasympathetic nervous system acquired from the subject, and a mechanism for quantifying and holding the captured information as the biological data. Note that, the bio-information of the sympathetic nervous system and the parasympathetic nervous system may be acquired separately by different methods from different living body parts (different parts of the body) or the same living body part.

The biological data analysis unit 22 includes a mechanism for judging, based on the biological data acquired by the biological data acquisition unit 21, candidate group of sensitivity factors that the subject has. The biological data analysis unit 22 may judge the biological data obtained by acquiring the reaction of the sympathetic nervous system and the biological data obtained by acquiring the reaction of the parasympathetic nervous system in combination to extract a candidate group of sensitivity factors, or may judge the biological data of the sympathetic nervous system and the biological data of the parasympathetic nervous system separately to extract candidate groups of sensitivity factors and send the respective candidate groups of sensitivity factors to the comprehensive evaluation device 4. Alternatively, the biological data analysis unit 22 may extract a candidate group of sensitivity factors depending on degrees of reactions of the sympathetic nervous system and the parasympathetic nervous system obtained from the subject. Yet alternatively, the biological data analysis unit 22 may extract candidate groups of sensitivity factors based on biological data obtained from the subject at a plurality of timings, which are different in time. In this case, along and in association with the extracted candidates of sensitivity and the degrees thereof at the respective timings, mean values, maximum values, minimum values, and the like of the degrees of the reactions of the respective nervous systems in the measurement periods may also be sent to the comprehensive evaluation device 4.

The positive/negative evaluation device 3 includes a PN measurement unit 31 and a PN analysis unit 32.

The PN measurement unit 31 includes a mechanism for capturing information required for estimating pleasantness/unpleasantness of the subject based on text information, an image, a voice, sensors, and the like obtained from the subject via the input/output device 1, and a mechanism for quantifying and holding the captured information.

The PN analysis unit 32 includes a mechanism for judging, based on the information acquired by the PN measurement unit 31, whether an inner state that the subject has is pleasant or unpleasant. Alternatively, the PN analysis unit 32 includes a mechanism for expressing the inner state that the subject has in multiple levels ranging from pleasant to unpleasant, and judging, based on the information acquired by the PN measurement unit 31, which of the levels the inner state is at.

Note that, the PN analysis unit 32 may use for the judgment a plurality of kinds of information obtained from the subject. At this time, it is more preferred to acquire, along with the plurality of kinds of information obtained from the subject, information for analyzing the pleasantness or unpleasantness at a plurality of timings. Of course, a single kind of information on the subject may be acquired and judged at a plurality of timings.

The comprehensive evaluation device 4 operates as control means for controlling and regulating the operation of the sensitivity evaluation system as a whole. In addition, the comprehensive evaluation device 4 includes a mechanism for integrating analysis results of the biological data evaluation device 2 and the positive/negative evaluation device 3 to estimate the sensitivity and its intensity of the subject.

The comprehensive evaluation device 4 may estimate the sensitivity of the subject by a method of extracting, from among the candidate group of sensitivity factors obtained from the biological data analysis unit 22, sensitivity corresponding to positive or negative in the PN analysis unit 32. Alternatively, when the multiple levels ranging from positive to negative may be acquired, a sensitivity candidate corresponding to the level may be extracted.

The comprehensive evaluation device 4 may additionally include a mechanism for extracting one or a plurality of sensitivity candidates and the degrees thereof in association with each of the reaction of the sympathetic nervous system and the reaction of the parasympathetic nervous system, and calculating, from the extracted kinds of sensitivity and the degrees thereof, the “attractiveness” and the like that the subject felt during the evaluation.

Next, the overall operation according to this embodiment is described in detail.

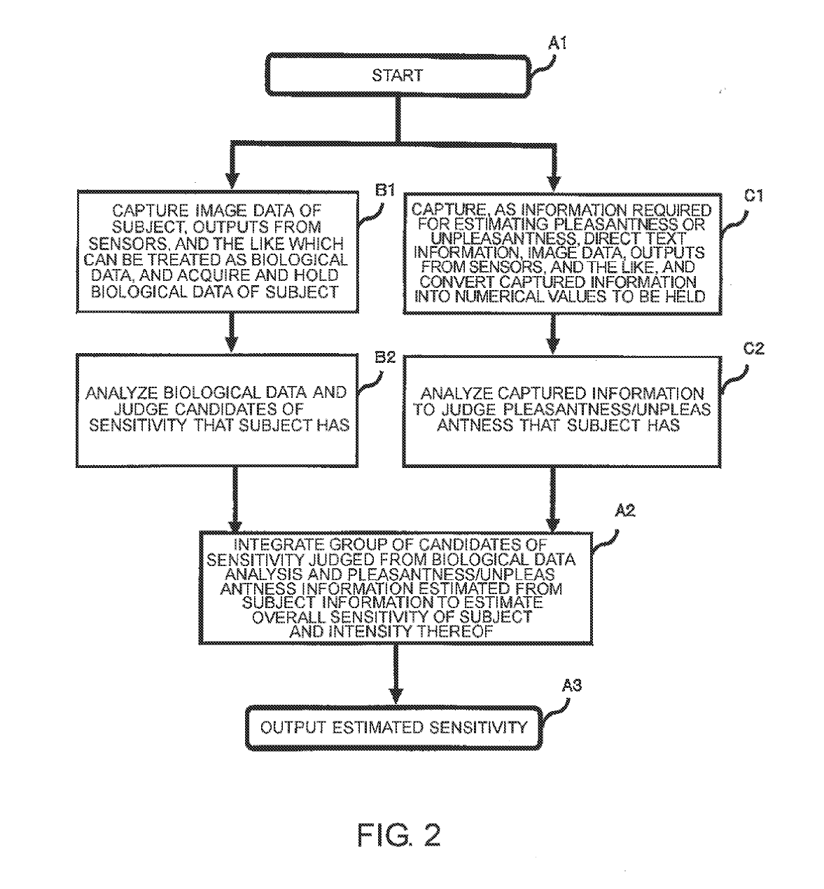

FIG. 2 is a flowchart illustrating a flow of processing of the sensitivity evaluation system.

The comprehensive evaluation device 4 instructs the biological data evaluation device 2 and the positive/negative evaluation device 3 to start acquiring the information required for estimating the sensitivity and its intensity of the subject (Step A1).

The biological data acquisition unit 21 of the biological data measurement device 2 captures the image of the subject, outputs from various sensors, and the like, which can be treated as the bio-information, via the input/output device 1 and converts the reaction of the sympathetic nervous system and the reaction of the parasympathetic nervous system into data as the biological data (Step B1).

The biological data analysis unit 22 performs analysis processing on the biological data acquired by the biological data acquisition unit 21, and sends candidates of the sensitivity that the subject has to the comprehensive evaluation device 4 (Step B2).

Simultaneously with Steps B1 and B2 described above, the PN measurement unit 31 of the positive/negative evaluation device 3 captures, as the information required for judging the pleasantness/unpleasantness, direct text information, image data, outputs from the sensors, and the like, and converts the captured information into data to be held (Step C1).

The PN analysis unit 32 analyzes the data acquired by the PN measurement unit 31 to judge the pleasantness or unpleasantness that the subject has, and its level when necessary, and sends the result to the comprehensive evaluation device 4 (Step C2).

The comprehensive evaluation device 4 acquires the analysis results from the biological data evaluation device 2 and the positive/negative evaluation device 3, respectively, and judges, from among the candidates of sensitivity of the subject, a result corresponding to the pleasantness or unpleasantness and the degree thereof as the sensitivity of the subject to be recorded in a storage unit (Step A2). Note that, at this time, the “attractiveness” of the target that caused a change in the sensitivity of the subject may be calculated.

The comprehensive evaluation device 4 outputs the estimated sensitivity of the subject via the input/output device 1 (Step A3).

As described above, according to this embodiment, the positive/negative judgment is performed along with the analysis of the biological data, and the results are matched with each other. Therefore, a subject sensitivity evaluation method of extracting accurate potential sensitivity and the intensity thereof may be provided.

Next, this invention is described by way of a second embodiment.

In this embodiment, the positive/negative judgment is different from that of the configuration described above.

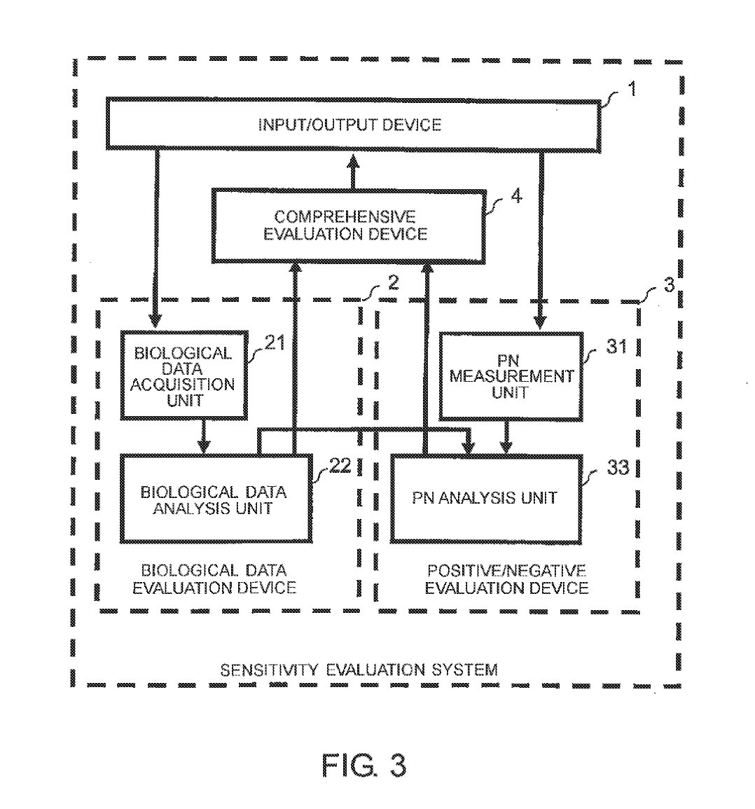

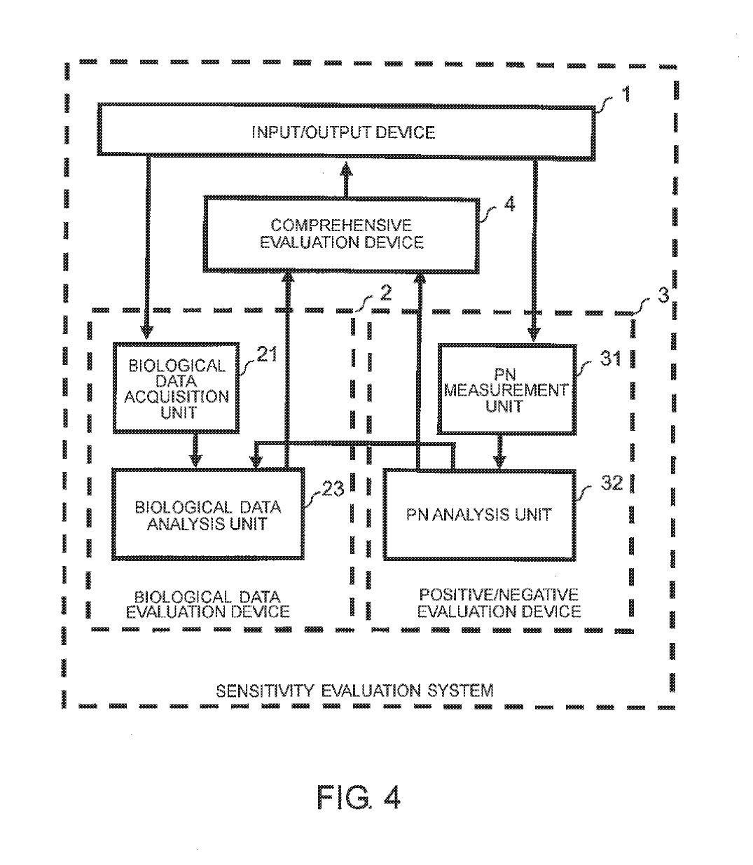

FIGS. 3 and 4 are block diagrams illustrating configurations according to the second embodiment of this invention.

The positive/negative evaluation device 3 illustrated in FIG. 3 acquires the biological data of both or one of the sympathetic nervous system and the parasympathetic nervous system from the biological data evaluation device 2, and matches the biological data with the judgment result of pleasantness and unpleasantness to determine the reliability of the judgment result. The reliability is used in the comprehensive evaluation device 4.

From each piece of the biological data acquired by the biological data evaluation device 2, the intensities of the reactions of the sympathetic nervous system and the parasympathetic nervous system of the subject may be read. Therefore, by matching the times when the positive/negative judgment was performed with the change in reactions of the sympathetic nervous system and the parasympathetic nervous system at the times, the reliability of the positive/negative judgment is improved. Of course, a measurement delay until the intensities of the reactions of the sympathetic nervous system and the parasympathetic nervous system appear is considered.

In the configuration illustrated in FIG. 3, the judgment is performed by a PN analysis unit 33. However, the validation of the reliability of the judgment may be performed in the biological data evaluation device 2 as illustrated in FIG. 4.

In this case, a biological data analysis unit 23 acquires the judgment result of pleasantness and unpleasantness from the positive/negative evaluation device 3, and matches the judgment result with the biological data of both or one of the sympathetic nervous system and the parasympathetic nervous system to determine the reliability of the judgment result.

An example of the validation of the reliability of the above-mentioned positive/negative judgment result is described below. In this example, a mean value of pupil diameters during a period is used to measure the “intensity of interest/intensity of stress” from the sympathetic nervous system and extract the sensitivity of the “degree of relaxation/degree of fatigue” from the parasympathetic nerve. In this case, the positive/negative evaluation is performed during the above-mentioned period, and based on the result of the evaluation, the comprehensive evaluation device 4 classifies the sensitivity.

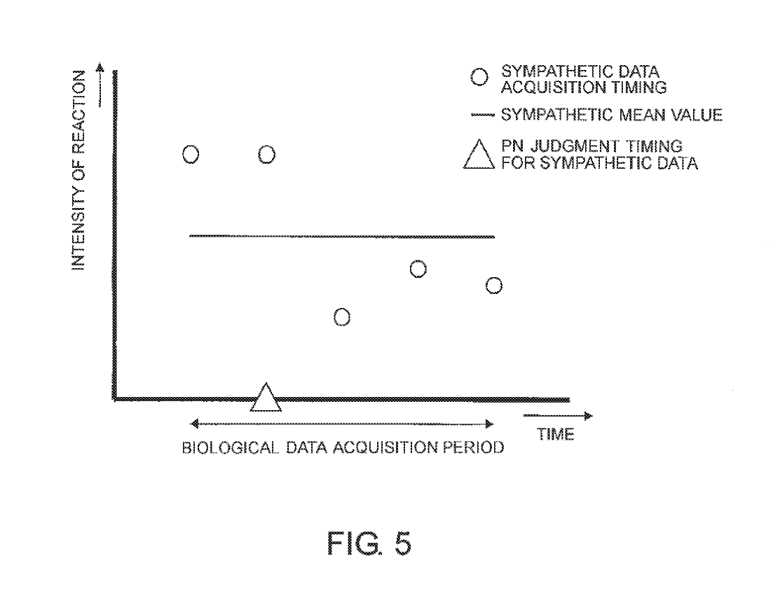

FIG. 5 is an explanatory diagram illustrating how the intensity of the reaction of the sympathetic nervous system is used to validate the reliability of the positive/negative judgment. Note that, for simplicity of the description, only the sympathetic nervous system is described. The validation of the reliability also works effectively when the sympathetic nervous system and the parasympathetic nervous system are used in combination, or when only the parasympathetic nervous system is used. As illustrated in FIG. 5, in order to acquire the candidate group of sensitivity factors, the biological data evaluation device 2 acquires data of the pupil diameters for a predetermined period (white circles in the graph). From the mean of the pupil diameters, the intensity that the candidate group of sensitivity factors has during the period is calculated. At the same time, the positive/negative evaluation device 3 performs the positive/negative judgment (white triangle in the graph).

At this time, in the positive/negative judgment, it is judged from information obtained from the PN measurement unit 31 whether the subject is pleasant or unpleasant. Along with the judgment, a magnitude of the biological data obtained from the pupil diameter at the time of acquiring the information used in the judgment is referred to. As the magnitude, a mean value, a maximum value, a variation, or the like may be used as appropriate depending on the kind of sensitivity. When the result for the obtained sensitivity is “high”, and when the result for the value indicated by the pupil diameter at the time of the judgment is “high”, the reliability is rated high.

On the other hand, even when the result for the obtained sensitivity is “high”, when the value of the pupil diameter at the time of performing the positive/negative evaluation indicates “low”, the reliability is rated low.

Note that, it is also possible to align the timing at which the data for performing the positive/negative evaluation is acquired, and the timing at which the autonomic biological data for use in extracting the candidate group of sensitivity factors is acquired. Further, for each candidate group of sensitivity factors to be extracted, the timings, the frequency, and the kind of data to be collected may be switched.

In the above description, the validation of the reliability of the positive/negative judgment result is performed by the biological data analysis unit 23 or the PN analysis unit 33, but the validation may be performed in the comprehensive evaluation device 4.

As described above, according to this embodiment, the positive/negative judgment is judged for reliability and matched with the analysis result of the biological data. Therefore, the subject sensitivity evaluation method of extracting the accurate potential sensitivity and the intensity thereof may be provided.

Next, this invention is described by way of yet another embodiment.

In this embodiment, a feedback is provided to the analysis of the biological data and the positive/negative judgment based on the sensitivity factors judged by the comprehensive evaluation device, to thereby improve the accuracies thereof separately or integratively.

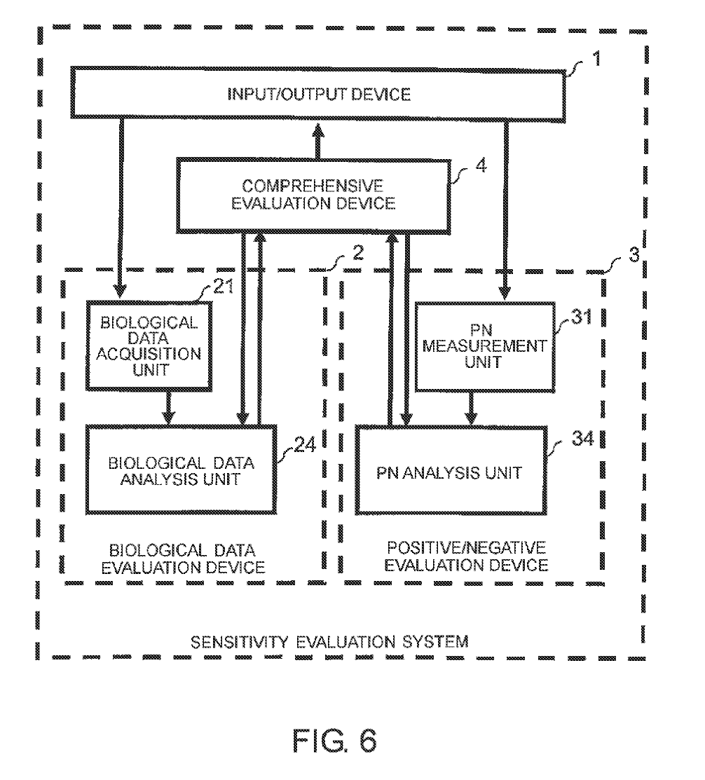

FIG. 6 is a block diagram illustrating a configuration according to a third embodiment of this invention.

A biological data analysis unit 24 according to this embodiment receives the estimated sensitivity factors from the comprehensive evaluation device 4, and judges, from the estimated sensitivity factors and the biological data obtained from the biological data acquisition unit 21, the candidate group of sensitivity factors with high accuracy.

As a feedback, if the effects of both the sympathetic nervous system and the parasympathetic nerve are reflected on the first judgment of the candidate group of sensitivity factors, the effect of one of the sympathetic nervous system and the parasympathetic nerve is corrected for the next and subsequent judgments of the candidate group of sensitivity factors. As another feedback, the accuracy and method of acquiring the biological data and the measurement device are adapted to suit the acquisition of the sensitivity factors estimated by the comprehensive evaluation device 4.

Thereafter, the candidate group of sensitivity factors judged based on the reacquired biological data is sent to the comprehensive evaluation device 4.

On the other hand, a PN analysis unit 34 receives the judged sensitivity factors from the comprehensive evaluation device 4, and judges the inner state of the subject with high accuracy from the estimated sensitivity factors and the information obtained from the PN measurement unit 31.

The feedback may be provided similarly to the biological data analysis unit 24.

Note that, the above-mentioned feedback may be provided to only one of the biological data analysis unit 24 and the PN analysis unit 34.

As described above, according to this embodiment, the sensitivity factors estimated by the comprehensive evaluation device 4 are fed back in evaluating the judgments, and hence the reliability and accuracy of each of the judgments are improved. As a result, the sensitivity evaluation method improved in the reliability and accuracy of the final sensitivity estimation may be provided.

### EXAMPLE

Next, the operation of this invention is described by way of a specific example.

In this example, a new model of a mobile phone is handed to the subject, and a degree of attractiveness of the new model is measured. Further, a case is considered where, as the biological data evaluation device 2, one or a plurality of non-contact, cap-type, glass-type, or wristband-type portable measuring apparatus are used as needed to measure the biological data regarding the sympathetic nervous system/parasympathetic nervous system that can be measured non-invasively.

Sources of the bio-information may include the pupil diameter, the temperature near a nostril, and the like, but this invention is not limited thereto. Other sources may include the brain wave, the pulse wave from a finger or the like, a respiration rate, sweating, a change in blood pressure, and the like.

Further, sources of the positive/negative analysis may include contents of answers to the questions asked to the subject, the image taken of the subject, the voice of the subject, the gesture of the subject, and the like.

The biological data acquisition unit acquires, for the pupil diameter, for example, image data of an eye of the subject via image capturing means. For the temperature near the nostril, for example, a thermography is used to measure and acquire the temperature by specifying a certain area around the nose on the face. For others, the existing methods of acquiring biological data, such as brain wave measurement and pulse wave measurement, may be used. Further, those pieces of bio-information may be acquired collectively for both the sympathetic nervous system and the parasympathetic nervous system, but are acquired separately in this example. As an example, the pupil diameter may be acquired for the sympathetic nervous system, and the temperature near the nostril may be acquired for the parasympathetic nervous system. As another example, for example, the sympathetic nervous system may be measured by fingertip sphygmogram, and the parasympathetic nervous system may be measured by alpha wave measurement of the brain wave.

For the pupil diameter, the biological data analysis unit calculates, for example, an average normalized score of the pupil diameters in a predetermined period of time, and judges, based on the magnitude of the average normalized score, the reaction of the parasympathetic system and the degree thereof. For the temperature near the nostril, the biological data analysis unit calculates, for example, a difference between the highest temperature and the lowest temperature during the predetermined period of time, and judges, based on the magnitude of the difference, the reaction of the sympathetic nervous system. Alternatively, the reaction of the sympathetic nervous system may be acquired from the pupil, or a respiration frequency or the like may be used as a measure for measuring the magnitude of the reaction.

Note that, the bio-information is collected by a portable electroencephalograph or a watch-type pulsimeter, and hence the sensitivity felt by the subject about the atmosphere at the time or about the target may be estimated even when the subject is moving. As a result, calculation of the attractiveness and the like may be performed with high accuracy. This enables the attractiveness to be measured during an activity or outdoors, for example.

The biological data analysis unit may judge, by referring to the sympathetic nervous system, the magnitude and the intensity of “desire”, “interest”, “excitement”, “stress”, and the like of the current inner states of the subject. Further, the biological data analysis unit may judge, by referring to the parasympathetic nervous system, the magnitude and the intensity of “feeling of security”, “relaxation”, “fatigue”, and the like.

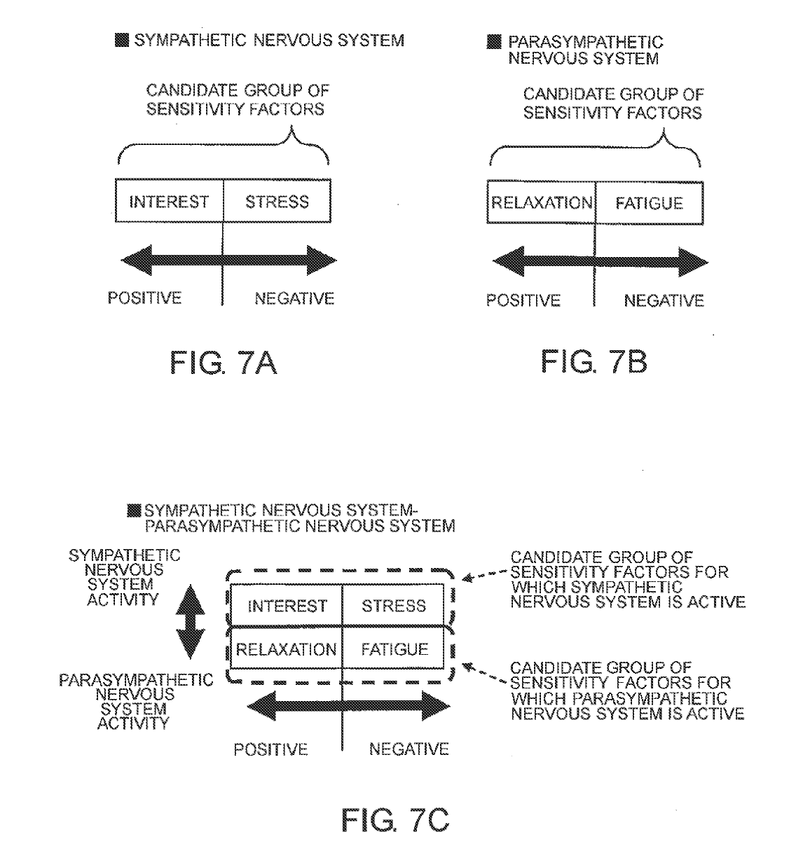

Next, examples of the candidate groups of sensitivity factors to be judged are illustrated in FIGS. 7A to 7C. FIG. 7A illustrates an example in which the biological data analysis unit judges a set of “interest and stress” as the candidate group of sensitivity factors. In the judgment, the candidate group of sensitivity factors may be selected from a number of candidate groups of sensitivity factors based on the biological data of the sympathetic nervous system, or may be extracted by another method. FIG. 7B illustrates an example of the candidate group of sensitivity factors judged in the analysis by the parasympathetic nervous system. FIG. 7C illustrates an example in which, based on a judgment obtained by a combined analysis of the sympathetic nervous system and the parasympathetic nervous system, the candidate group of sensitivity factors that is more active is selected. As illustrated in FIGS. 7A to 7C, the candidate group of sensitivity factors includes at least two kinds of sensitivity factors ranging from positive to negative in association with each other.

Note that, a plurality of sensitivity factors are mixed in the reactions (magnitude, action, and the like) of the sympathetic nervous system and the parasympathetic nervous system that can be acquired from the living body parts (measurement targets) via the respective portable measurement apparatus. Further, many living body parts are under dual control of the sympathetic nervous system and the parasympathetic nervous system.

Further, when the pupil diameter contracts, the parasympathetic nervous system may be said to be active. However, it cannot be judged whether the parasympathetic nervous system is active from the “feeling of security” or the parasympathetic nervous system is active from an accumulation of the “fatigue”. Similarly, when the pupil diameter dilates, it cannot be judged from the fact that the sympathetic nervous system is active whether the sensitivity causes the “desire” for the product when, for example, the subject is operating the product, or the subject is feeling the “stress” from the operability of the product and the like.

According to this invention, the comprehensive evaluation device 4 classifies the sensitivity factors by referring to the positiveness/negativeness of the subject.

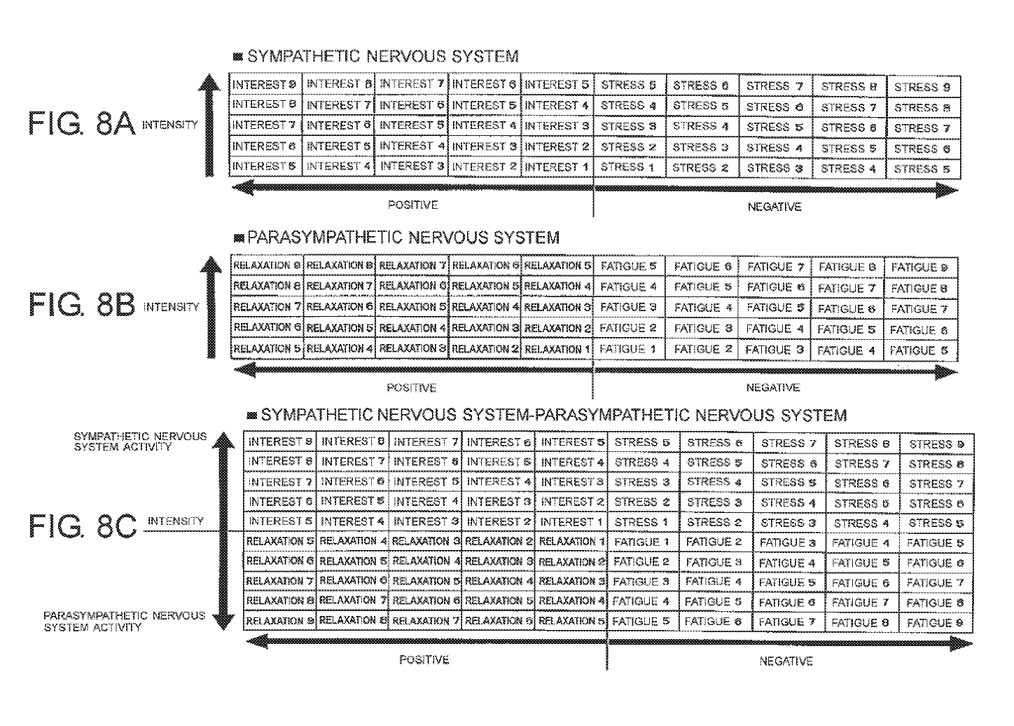

The candidate group of sensitivity factors illustrated in FIGS. 7A to 7C are classified by the comprehensive evaluation device 4 based on whether each of the sensitivity factors is positive or negative, and are used in the final sensitivity estimation. As an alternative thereto, the comprehensive evaluation device 4 may be caused to select from the candidate group of sensitivity factors a sensitivity factor corresponding to a degree of positiveness and negativeness. In this case, as illustrated in FIGS. 8A to 8C, a plurality of sensitivity factors may be arranged (in the figures, 10 types in the horizontal direction) to be associated with degrees of positiveness and negativeness, to thereby obtain one set of candidate groups of sensitivity factors.

Further, in the examples of FIGS. 8A to 8C, a plurality of sensitivity factors are arranged also for degrees of the activities of the sympathetic nervous system and the parasympathetic nervous system (in the figure, 5 or 10 types in the vertical direction). The tables illustrated in FIGS. 8A and 8B are used when the sensitivity factors are judged separately for the sympathetic nervous system and the parasympathetic nervous system, respectively. The table illustrated in FIG. 8C is used when the sensitivity factors are judged for the sympathetic nervous system and the parasympathetic nervous system in combination. In this table, the candidate group of sensitivity factors to be selected changes depending on the degrees of activities of the sympathetic nervous system and the parasympathetic nervous system. This means, for example, that the candidate group of sensitivity factors to be selected is different for a case where the sympathetic nerve has high activity and the parasympathetic nerve has some activity, and a case where the sympathetic nerve has high activity and the parasympathetic nerve is inactive. Therefore, the sensitivity evaluation system may extract the sensitivity of the subject with higher accuracy.

The positive/negative evaluation device 3 extracts complementary information for complementing the judgment on whether each of the reactions of the sympathetic nervous system and the parasympathetic nervous system obtained by the biological data evaluation device 2 is positive (pleasant direction) or negative (unpleasant direction). The sensitivity evaluation system acquires the complementary information in association with the degrees of positiveness and negativeness when necessary.

When the positive/negative evaluation device 3 extracts the pleasantness/unpleasantness from a questionnaire to the subject, for example, the PN measurement unit 31 asks questions in view of “imposing little load on the subject”, “causing no discomfort”, and the like, and stores answers thereto. The answers to the questions are easily made by being recognized as “a touch on a predetermined position on a predetermined touch panel or the like”, “a movement of a predetermined object”, “a change in orientation”, “a predetermined operation”, and the like. Examples of the questions may be “For positive, place the object on the right”, “For positive, place the object vertically”, and “For positive, shake the object”.

The PN analysis unit 32 analyzes the answers to the questions to the subject, and judges whether the feeling of the subject is in the pleasant direction or the unpleasant direction with two levels or with multiple levels.

Further, for extracting the pleasantness/unpleasantness from the facial expression of the subject, for example, the PN measurement unit 31 may acquire the shape or motion of a muscle of facial expression, an image of the facial expression as a whole, and the like at the time of measurement, and the PN analysis unit 32 may judge, by subjecting the shape and motion of the muscle of facial expression and the facial expression as a whole to the pattern recognition or the like, whether the feeling of the subject is in the pleasant direction or the unpleasant direction with the two levels or with the multiple levels.

The comprehensive evaluation device 4 integrates the result obtained from the biological data evaluation device 2 and the result obtained from the positive/negative evaluation device 3 to make a comprehensive evaluation.

The biological data regarding the sympathetic nervous system has, when the sympathetic nervous system is activated, opposite poles of a positive state such as “interested” and a negative state such as “highly stressed”.

Similarly, the biological data regarding the parasympathetic nervous system has, when the parasympathetic nervous system is activated, opposite poles of a positive state such as “relaxed” and a negative state such as “severely fatigued”.

As a complement thereto, the comprehensive evaluation device 4 acquires the judgment result regarding the positiveness or negativeness and, when necessary, the degree thereof (collectively referred to as complementary information) from the positive/negative evaluation device 3, and selects the corresponding sensitivity factor from the candidate group of sensitivity factors, which is output from the biological data measurement device 2 as a result of the judgment of the autonomic nerve, to thereby estimate the sensitivity comprehensively. Thereafter, a feedback is provided to the biological data evaluation device 2 and the positive/negative evaluation device 3 as appropriate, to thereby improve the reliability of the estimated sensitivity. For example, the sensitivity evaluation system may operate, when estimating in the estimation of the sensitivity regarding the sympathetic nervous system and the parasympathetic nervous system that the parasympathetic nervous system is more active than the sympathetic nervous system and the result indicates the positive evaluation of a relaxed state, to acquire the biological data of the parasympathetic nervous system via measurement equipment suitable for measuring the degree of the relaxed state.

Further, the biological data analysis unit may judge, from the biological data obtained from each of the living body parts, the relaxed state that the subject has, again in consideration of the degree of activity of the sympathetic nervous system in addition to the parasympathetic nervous system. Further, the sensitivity evaluation system may operate to perform the validation by a plurality of pieces of measurement equipment in consideration of the measurement accuracy for the sensitivity factor of the measurement equipment which has acquired the reactions from the living body parts. With this configuration, the comprehensive evaluation device 4 may acquire the sensitivity regarding only the sympathetic nervous system, the sensitivity regarding only the parasympathetic nervous system, and the sensitivity regarding the sympathetic nervous system and the parasympathetic nervous system with high accuracy as necessary.

Further, the degree of attractiveness (attractive, want to buy one, want one, want to use one, and the like) of the mobile phone is calculated based on the sensitivity candidates and the degrees thereof estimated from the reaction of the sympathetic nervous system and the reaction of the parasympathetic nervous system.

The calculation of the attractiveness may be defined by connections and interdependence of factors obtained by dividing the definition of attractiveness.

As an example, one of major factors in the attractiveness of the mobile phone is an item related to the operation, such as “understandability”, “practicality”, and “controllability”. The “comfortableness” and “feeling of security” are also important.

Another major factor in the attractiveness is an item induced by a desire or an interest, such as “fun”, “excitement”, “want to touch the phone”, and “want to show the phone to others”.

The factors are named “operability factor” and “motivation factor”, respectively. Factors belonging to the operability factor and the motivation factor are scored in association with the sensitivity estimated by the comprehensive evaluation device 4 to calculate the final value of attractiveness.

Note that, the motivation factor tends to reflect the reaction of the sympathetic nervous system. The operability factor tends to reflect the reaction of the parasympathetic nervous system. When the biological data analysis unit 22 analyzes the sympathetic nervous system and the parasympathetic nervous system separately and sends the respective results to the comprehensive evaluation device 4, the sensitivity evaluation system may score the operability factor and the motivation factor as well as the overall attractiveness. As a result, the sensitivity evaluation system may display, from the reactions of the subject, a chart in association with the intrinsic factors. Further, the sensitivity evaluation system may cause the subject to operate a plurality of models to display the difference in attractiveness among the models felt by the subject as a chart.

Note that, the calculation of the attractiveness works effectively when the factors that can lead to opposite evaluations are classified by the positive/negative judgment.

Further, the sensitivity evaluation system can be used in, for example, a terminal suggestion system for displaying, depending on the kind of the judged sensitivity and the sensitivity, a terminal that the subject wants from the potential sensitivity as a recommended terminal.

Similarly, by using the evaluation result from the system of this invention, there may be provided, for example, a Web system for assigning, when an estimation can be made that the subject is more attracted to a content, high evaluation points to the content, and operating so as to arrange the content in a manner that is easily accessible or to present contents similar to the content as recommendations the next and subsequent times.

Note that, as can be understood from the above description, for factors for which the sensitivity of the subject can be extracted from one of the biological data evaluation device 2 and the positive/negative evaluation device 3 with sufficient accuracy, matching the analysis results of the biological data evaluation device 2 and the positive/negative evaluation device 3 may be omitted.

Further, the devices (units) of the sensitivity evaluation system may be realized by using a combination of hardware and software. In a mode that combines hardware and software, the units are realized as various means by deploying a sensitivity evaluation program to a RAM and operating hardware such as a control unit (CPU) based on the program. Further, the program may be distributed by being recorded on a recording medium. The program recorded on the recording medium is read to a memory via wire, wirelessly, or via the recording medium itself to operate the control unit and the like. Note that, examples of the recording medium may include an optical disc, a magnetic disk, a semiconductor memory device, and a hard disk.

When the above-mentioned embodiments are expressed differently, the information processing apparatus that is caused to operate as the sensitivity evaluation system may be realized by causing, based on the sensitivity evaluation program deployed to the RAM, hardware to operate as the comprehensive evaluation device, the biological data evaluation device, and the positive/negative evaluation device.

As described above, the sensitivity evaluation system to which this invention is applied may extract the potential sensitivity without any intention.

Further, the sensitivity that the subject pretends to have may also be detected by referring to the autonomic nervous system.

In addition, the classification of the candidate group of sensitivity factors obtained from the bio-information, which cannot be performed with the existing technologies, may be performed. Similarly, from among the candidate group of potential sensitivity factors, accurate sensitivity may be extracted depending on the degree of positiveness and negativeness.

Note that, the specific configurations of this invention are not limited to the embodiment modes and the example described above, and this invention encompasses modifications without departing from the gist of this invention. For example, the above-mentioned first to third embodiments may be combined as appropriate for operation.

According to this invention, the potential sensitivity without any intention to a system or a service may be extracted by a flexible evaluation environment and fed back to the development of the system or the service.

This application claims priority from Japanese Patent Application No. 2010-138556, filed on Jun. 17, 2010, the entire disclosure of which is incorporated herein by reference.

### REFERENCE SIGNS LIST

1 input/output device (input/output means)
2 biological data evaluation device
21 biological data acquisition unit (biological data acquisition means)
22, 23, 24 biological data analysis unit (biological data analysis means)
3 positive/negative evaluation device
31 PN measurement unit
32, 33, 34 PN analysis unit (PN analysis means)
4 comprehensive evaluation device

1. A sensitivity evaluation system, comprising:
an acquisition unit to acquire as biological data a reaction of a sympathetic nervous system and/or a reaction of a parasympathetic nervous system;
a biological data analysis unit to judge from the obtained biological data, a candidate group of sensitivity factors that a subject has;
a positive/negative analysis unit to judge from information obtained from the subject, whether an inner state of the subject is a positive pleasant state or a negative unpleasant state; and
a comprehensive evaluation unit to integrate analysis results of the biological data analysis unit and the positive/negative analysis unit to estimate sensitivity comprehensively,
wherein:
the candidate group of sensitivity factors are candidate groups associated with an active degree of the sympathetic nervous system and/or the parasympathetic nervous system,
wherein:
the candidate groups include a plurality of sensitivity factors in the form of a set of positive and negative sensitivity factors.

2. A sensitivity evaluation system according to claim 1, wherein the acquisition unit acquires the reaction of the sympathetic nervous system and/or the reaction of the parasympathetic nervous system from different living body parts.

3. A sensitivity evaluation system according to claim 1, wherein the biological data analysis unit judges the biological data obtained by acquiring the reaction of the sympathetic nervous system and the biological data obtained by acquiring the reaction of the parasympathetic nervous system in combination to extract the candidate group of sensitivity factors.

4. A sensitivity evaluation system according to claim 1, wherein the biological data analysis unit judges the biological data obtained by acquiring the reaction of the sympathetic nervous system and the biological data obtained by acquiring the reaction of the parasympathetic nervous system separately to extract each candidate group of sensitivity factors.

5. A sensitivity evaluation system according to claim 1, wherein the biological data analysis unit judges, based on the biological data obtained from the subject at a plurality of timings, the candidate group of sensitivity factors that the subject has.

6. A sensitivity evaluation system according to claim 1, wherein the positive/negative analysis unit judges, based on a plurality of kinds of information for analyzing pleasantness and unpleasantness obtained from the subject, whether the subject is pleasant or unpleasant.

7. A sensitivity evaluation system according to claim 1, wherein the positive/negative analysis unit judges, based on information for analyzing pleasantness and unpleasantness obtained from the subject at a plurality of timings, whether the subject is pleasant or unpleasant.

8. A sensitivity evaluation system according to claim 1, wherein the positive/negative analysis unit matches a result of the judgment of pleasantness and unpleasantness and the acquired biological data of the sympathetic nervous system and/or the parasympathetic nervous system to determine a reliability of the result of the judgment.

9. A sensitivity evaluation system according to claim 1, wherein the comprehensive evaluation unit extracts, from the candidate group of sensitivity factors obtained from the biological data analysis unit, corresponding sensitivity based on a result of the judgment obtained from the positive/negative analysis unit.

10. A sensitivity evaluation system according to claim 1, wherein the comprehensive evaluation unit extracts, from the candidate group of sensitivity factors obtained from the biological data analysis unit, corresponding sensitivity based on a degree of positiveness and negativeness included in a result of the judgment obtained from the positive/negative analysis unit.

11. A sensitivity evaluation system according to claim 1, wherein the comprehensive evaluation unit extracts, in association with each of the reaction of the sympathetic nervous system and the reaction of the parasympathetic nervous system, a kind of the sensitivity and a degree thereof, and calculates, based on the extracted kind of the sensitivity and the extracted degree thereof, an “attractiveness” that the subject felt during an evaluation.

12. A sensitivity evaluation system according to claim 1, wherein the biological data analysis unit uses, as the biological data acquired from the sympathetic nervous system and/or the parasympathetic nervous system, any one of a pupil diameter, a temperature near a nostril, and a brain wave.

13. A sensitivity evaluation system according to claim 1, wherein the positive/negative analysis unit judges, from any one or a combination of text information obtained as an answer to a question asked to the subject, image information taken of the subject, voice information obtained by acquiring a voice of the subject, and gesture information of the subject, whether the subject is pleasant or unpleasant, or a degree of pleasantness or unpleasantness.

14. A sensitivity evaluation system according to claim 1, wherein the sensitivity evaluation system sets, based on a judged kind of the sensitivity and/or a judged intensity of the sensitivity, an information processing operation to be performed next from among predetermined options of the information processing operation in association with kinds of the sensitivity and/or intensities of the sensitivity to be judged.

15. (canceled)

16. A sensitivity evaluation method, comprising:
acquiring a reaction of a sympathetic nervous system as biological data;
acquiring a reaction of a parasympathetic nervous system as the biological data;
performing judgment processing, from the obtained biological data, about a candidate group of sensitivity factors that candidate groups associate with an active degree of the sympathetic nervous system and/or the parasympathetic nervous system, and the candidate groups include a plurality of sensitivity factors in the form of a set of positive and negative sensitivity factors;
performing judgment processing, from information obtained from the subject, sensitivity factors on whether an inner state of the subject is the pleasant state or the unpleasant state; and
performing estimation processing, based on the judged pleasant state or unpleasant state, about sensitivity from among the candidate group of sensitivity factors obtained by the judgment processing.

17. (canceled)

18. A recording medium having recorded thereon a sensitivity evaluation program for causing a control unit of an information processing apparatus to operate as:
a biological data analysis unit to judge, from biological data obtained from an acquisition unit for acquiring a reaction of a sympathetic nervous system and a reaction of a parasympathetic nervous system, a candidate group of sensitivity factors that a subject has;
a positive/negative analysis unit to judge, from information obtained from the subject, whether an inner state of the subject is a pleasant state or an unpleasant state; and
a comprehensive evaluation unit to integrate analysis results of the biological data analysis unit and the positive/negative analysis unit to estimate sensitivity comprehensively,
wherein:
the candidate group of sensitivity factors are candidate groups associated with an active degree of the sympathetic nervous system and/or the parasympathetic nervous system,
wherein:
the candidate groups include a plurality of sensitivity factors in the form of a set of positive and negative sensitivity factors.
